(19) United States
(12) Patent Application Publication
(10) Pub. No.: US 2009/0035311 A1
(43) Pub. Date: Feb. 5, 2009

(54) Identification and use of prognostic and predictive markers in cancer treatment

(75) Inventors: Soonmyung Paik, Pittsburgh, PA (US); Chungyeul Kim, Wexford, PA (US)

Correspondence Address: VINSON & ELKINS L.L.P., First City Tower, 1001 Fannin Street, Suite 2300, HOUSTON, TX, 77002-6760, US

(73) Assignee: NSABP Foundation, Inc., Pittsburgh, PA (US)

(21) Appl. No.: 12/284,419

(22) Filed: Sep. 22, 2008

Related U.S. Application Data
(63) Continuation of application No. 11/300,869, filed on Dec. 15, 2005, now abandoned.
(60) Provisional application No. 60/636,169, filed on Dec. 15, 2004.
(60) Provisional application No. 60/698,112, filed on Jul. 11, 2005.
(60) Provisional application No. 60/717,485, filed on Sep. 14, 2005.

(51) Int. Cl. A61K 39/395; A61P 35/00; C12Q 1/02; G01N 33/50; C12Q 1/68; C40B 30/04

(52) U.S. Cl. 424/133.1; 424/138.1; 435/29; 435/7.92; 435/7.23; 435/6; 506/9

(57) ABSTRACT

The present invention provides a method of screening for markers useful in predicting the efficacy of the treatment of a specified cancer that includes: (a) constructing a tissue microarray from a tissue bank comprising multiple tissue samples that are annotated with clinical follow-up data; (b) labeling polynucleic acid probes specific for oncogenes or cancer-associated genes known to be potential amplicons; (c) performing fluorescent in situ hybridization analysis on the tissue microarray; and (d) correlating the result of the fluorescent in situ hybridization with the clinical follow-up data. In addition, the present invention provides a method of treating breast cancer that includes measuring the expression levels or amplification of HTPAP in a patient having breast cancer and then providing a patient having increased levels of HTPAP expression or HTPAP amplification with therapeutic quantities of at least one compound that interferes with the phosphatidic acid phosphatase activity of HTPAP. The present invention also encompasses a method of treating breast cancer that includes screening a breast cancer patient for amplification of the cMYC gene and then treating a patient having amplification of the cMYC gene with therapeutic quantities of a compound that interferes with HER2 signaling.

### REFERENCE TO RELATED APPLICATION

This application is a continuation of U.S. patent application Ser. No. 11/300,869, filed on Dec. 15, 2005, which claims priority to U.S. Provisional Application Ser. Nos. 60/636,169, filed Dec. 15, 2004, 60/698,112, filed Jul. 11, 2005, and 60/717,485, filed Sep. 14, 2005, each of which are herein incorporated by reference in their entirety.

### BACKGROUND OF THE INVENTION

Breast cancer is a heterogeneous disease with respect to clinical behavior and response to therapy. This variability is a result of the differing molecular make-up of cancer cells within each subtype of breast cancer. However, despite recent advances in molecular taxonomy of breast cancer, only two molecular characteristics are currently being exploited as therapeutic targets. These are estrogen receptor (“ER”) and HER2, which are targets of antiestrogens (tamoxifen and aromatase inhibitors) and HERCEPTIN® respectively. Efforts to target these two molecules have proven to be extremely productive. Nevertheless, those tumors that do not have these two targets are often treated with chemotherapy, which generally targets proliferating cells. Since some important normal cells are also proliferating, they are damaged by chemotherapy at the same time. Therefore, chemotherapy is associated with severe toxicity. Identification of molecular targets in tumors in addition to ER or HER2 is critical in the development of new anticancer therapy.

Recent studies using a combination of cDNA array based expression profiling and comparative genomic hybridization (“CGH”) have elucidated the role of gene amplification in the transcriptional program of breast cancer. Copy number alteration and expression levels across 6691 mapped human genes were examined in 44 locally advanced breast cancer and 10 breast cancer cell lines (Pollack et al., Proc. Natl. Acad. Sci. USA 99(20):12963-12968, 2002). The data from this study suggests that at least 12% of all the variation in gene expression among the breast cancer samples is directly attributable to underlying variation in gene copy numbers. The total number of genomic alterations (gains and losses) correlated significantly with high grade (p=0.008), negative ER (p=0.04), and p53 mutation (p=0.0006). Of 117 high level amplifications (representing 91 different genes), 62% (representing 54 genes) were found to be associated with at least moderately elevated mRNA levels, and 42% (representing 36 different genes) with highly elevated mRNA levels. In a similar effort, the correlation between copy number changes and expression levels were examined in 14 breast cancer cell lines using cDNA microarray of 13,824 genes (Hyman et al., Cancer Res. 62(21):6240-6245, 2002). This study found 44% of highly amplified genes resulted in overexpression with 10.5% of overexpressed genes being amplified.

Together these results indicate a profound role of gene amplification in transcriptional control of gene expression in breast cancer and provide rationale for pursuing amplified genes as a preferred target for developing therapeutics and diagnostics. Unfortunately, no study has correlated clinical outcome with a comprehensive list of amplified genes in breast cancer although amplification of a handful of genes has been identified by array CGH and have been examined by fluorescence in situ hybridization (“FISH”) and found to be prognostic. The biggest barrier for the screening of amplification pattern is the cost and need for high quality DNA for array CGH assays.

On the other hand, FISH is a stable method that works with formalin-fixed paraffin-embedded sections in a routine clinical setting. FISH probes for HER2 have been FDA approved as a predictive test for HERCEPTIN® response. Due to the stability of DNA in the paraffin-embedded sections, it is more reliable than RNA-based or immunohistochemistry-based clinical assays. However, FISH probes for potentially important amplified genes have not been comprehensively developed. In fact, there is only one vendor (Vysis, Incorporated, Downers Grove, Ill.) that supplies an array of probes, but most of these probes have not been clinically validated at this point as prognostic factors. These probes are also very expensive (cost about $300 per case) and of limited variety, barely scratching the repertoire of potentially important amplicons in solid tumors such as breast and colon cancer.

In a recent survey of five Vysis-supplied commercial FISH probes (HER2, MDM2, MYC, CCND1, and EGFR) for potentially presumed important amplicons in breast cancer in 1100 cases, some but not all the five gene amplifications were found to correlate with survival outcome in a poorly defined clinical cohort with no treatment information (Al-Kuraya et al., Cancer Res. 64(23):8534-8540, 2004). Nevertheless, 60% of the cases did not have any amplification of the five genes examined. In addition, a gene amplification dosage effect was found in which survival rate was in the following order; no amplification >1 amplified>2 amplified>3 amplified. This data supports the so-called “amplificatory” phenotype with an increased level of genomic instability and high likelihood for amplification development, and therefore supports the need for a comprehensive clinical correlation of amplicons in breast cancer.

Approximately 15 to 20% of all breast cancers have overexpression of HER2 protein on its cell surface (Paik et al., J. Clin. Oncol. 8(1):103-112, 1990). Such tumors are known to have a worse prognosis than those without HER2 protein overexpression (Paik et al., 1990, supra). Overexpression of HER2 protein is almost invariably due to amplification or increased copy number of the gene encoding HER2.

Multiple drugs have been developed to target HER2 signaling as a means to stop growth of cancer cells that have overexpression of HER2 protein on their surface. One of these drugs is trastuzumab (HERCEPTIN®), developed by Genentech, Incorporated (South San Francisco, Calif.). HERCEPTIN® has recently been shown to be effective in prolonging survival in patients diagnosed with advanced breast cancer with HER2 overexpression (Slamon et al., N. Engl. J. Med. 344(11):783-792, 2001). Recently it has also been shown to reduce recurrences and death in patients with early stage breast cancer which have HER2 protein overexpression or HER2 gene amplification (Romond et al., N. Engl. J. Med. 353(16):1673-1684, 2005). The overall reduction in recurrence rate is about 50% with HERCEPTIN® when compared to chemotherapy alone in adjuvant setting (Romond et al., 2005, supra). Not all patients seem to gain benefit from this expensive treatment, which also has potential serious cardiotoxicity. A method to identify those patients who will benefit most from HERCEPTIN® or other HER2 targeting drugs is required (Slamon et al., 2001 supra; Goldman, J. Natl. Cancer Inst. 95(23):1744-1746, 2003). Many laboratories have been pursuing abnormalities in the components of the HER2 signaling pathway, such as PTEN, as predictors of response to HERCEPTIN®, with the hypothesis that such abnormalities will render tumor cells resistant to HERCEPTIN® even in the presence of HER2 protein overexpression (Crowder et al., Cancer Cell 6(2):103-104, 2004; Nagata et al., Cancer Cell 6(2):117-127, 2004). Such studies have concentrated only on molecules that may have a direct role in the HER2 signaling pathway, however, none have been substantiated in clinical studies, and there is no marker used for the prediction of response to HERCEPTIN® in clinical practice.

There are many genes that are amplified in breast cancer as demonstrated by CGH studies. As stated previously, about 10% of genes overexpressed in breast cancer are due to gene amplification (Pollack et al., 2002, supra). One of the frequently amplified genes in human cancers is cMYC, which is located on chromosome 8. In normal cells cMYC is expressed in a highly regulated manner driving cells from G1 to S phase. Perhaps due to its important role in normal cell proliferation, efforts to block cMYC have not been a major focus of the pharmaceutical industry. Only one company currently has a drug that is going through clinical testing (Cylene Pharmaceuticals, San Diego, Calif.). Studies have suggested that cMYC has an important role as a molecular switch that determines the cell's fate to go through programmed cell death or cell proliferation (Pelengaris et al., Nat. Rev. Cancer 2(10):764-776, 2002; Pelengaris et al., Cell 109(3):321-334, 2002). When cMYC is overexpressed, cells go into uncontrolled cell proliferation and become susceptible to programmed cell death in the absence of a survival signal (see FIG. 1a). cMYC induces apoptosis by regulating many components of the programmed cell death pathway, but the main effector seems to be Bax (Pelengaris et al., 2002, supra).

Eventually cells with cMYC overexpression will go through mass suicide due to the exhaustion of locally available survival factors. At the same time, cMYC overexpression has been shown to cause genomic instability. This could cause amplification of other oncogenes such as HER2 (Fest et al., Oncogene 21(19):2981-2990, 2002). Amplification of other genes could generate anti-apoptotic signals and therefore inhibit the apoptotic pathway. For example, in the case of HER2 amplification, studies have demonstrated that HER2 induces Bcl-2, an anti-apoptotic protein that inhibits Bax (Milella et al., Clin. Cancer Res. 10(22):7747-7756, 2004).

Nevertheless, a need remains to identify markers/genes that provide prognostic indicators of therapy efficacy. The references cited above and in the Appendix hereto are hereby incorporated by reference as if fully set forth herein.

### SUMMARY OF THE INVENTION

The present disclosure describes a new prognostic and therapeutic target, the HTPAP gene, which when amplified confers poor prognosis in breast cancer patients even after treatment with standard chemotherapy containing doxorubicin, cyclophosphamide, and paclitaxel. HTPAP amplification is an independent prognosticator of tumor size, treatment, number of positive axillary lymph nodes, age and hormone receptor status, HER2 amplification, and cMYC amplification. Furthermore, cMYC is a predictor of response to HERCEPTIN®, in such a way that for patients with cMYC amplification together with HER2 amplification/overexpression, there is a 75% reduction in cancer recurrence rate when HERCEPTIN® is added to chemotherapy, compared to only 45% reduction in recurrence rate for those patients without cMYC amplification. cMYC is amplified in approximately 30% of the breast cancer patients with HER2 amplification or overexpression. Inhibition of HER2 signaling by trastuzumab apparently changes the cMYC role from proliferation switch to pro-apoptotic switch. The invention has the following clinical applications: optimization of methods for patient selection and determining treatments using trastuzumab and other drugs that target a HER2 signaling pathway; optimization of methods for patient selection for future clinical studies that test the addition of other drugs or targeted therapies, such as bevacizumab (AVASTIN™) that targets angiogenesis, by allowing identification of patients who are at high risk of relapse even after trastuzumab or HER2 targeted therapy; a PCR-based assay that will detect the gene amplification status of both HER2 and cMYC in a single tube assay for prognostication and prediction of response in breast cancer patients; and rational development of cMYC targeted therapy through indirect modulation of its pro-apoptotic activity by inhibiting anti-apoptotic signals from other activated oncogenes.

### DESCRIPTION OF THE FIGURES

FIG. 1a shows a schematic of cMYC as a pro-apoptotic switch.

FIG. 1b shows a schematic of cMYC as a proliferation switch.

FIG. 1c shows a schematic of an anti-apoptotic signal from HER2.

FIG. 2 shows a flow chart describing a method of identifying therapeutic targets.

FIG. 3 shows the results of a clustering study.

FIG. 4 shows a chart of recurrence by amplification.

FIG. 5 shows a Kaplan-Meier plot for APPBP2.

FIG. 6 shows a Kaplan-Meier plot for BMP7.

FIG. 7 shows a Kaplan-Meier plot for bm_{—}009.

FIG. 8 shows a Kaplan-Meier plot for CACNB1.

FIG. 9 shows a Kaplan-Meier plot for chk.

FIG. 10 shows a Kaplan-Meier plot for c_myc.

FIG. 11 shows a Kaplan-Meier plot for cyclind1.

FIG. 12 shows a Kaplan-Meier plot for decr1.

FIG. 13 shows a Kaplan-Meier plot for FLJ 10783.

FIG. 14 shows a Kaplan-Meier plot for GRO1.

FIG. 15 shows a Kaplan-Meier plot for GRB2.

FIG. 16 shows a Kaplan-Meier plot for HBS1L.

FIG. 17 shows a Kaplan-Meier plot for HER2.

FIG. 18 shows a Kaplan-Meier plot for MAL2.

FIG. 19 shows a Kaplan-Meier plot for HTPAP.

FIG. 20 shows a Kaplan-Meier plot for MLN64.

FIG. 21 shows a Kaplan-Meier plot for MRPS7.

FIG. 22 shows a Kaplan-Meier plot for PPM1D.

FIG. 23 shows a Kaplan-Meier plot for NCO43.

FIG. 24 shows a Kaplan-Meier plot for RPS6KB1.

FIG. 25 shows a Kaplan-Meier plot for SEB4D.

FIG. 26 shows a Kaplan-Meier plot for stk6.

FIG. 27 shows a Kaplan-Meier plot for SIP2_{—}28.

FIG. 28 shows a Kaplan-Meier plot for TPD52

FIG. 29 shows a Kaplan-Meier plot for TRAF4.

FIG. 30 shows a Kaplan-Meier plot for ZNF217.

FIG. 31 shows a Kaplan-Meier plot for ZHX1.

FIG. 32 shows a Kaplan-Meier plot for any amplicon.

FIG. 33 shows a diagram of the HTPAP gene.

FIG. 34 shows a recurrence-free survival.

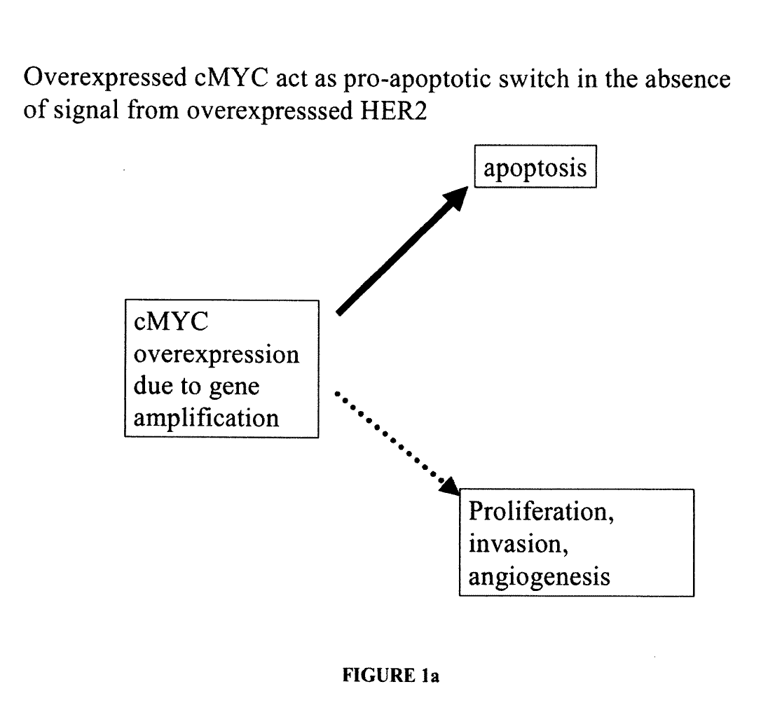

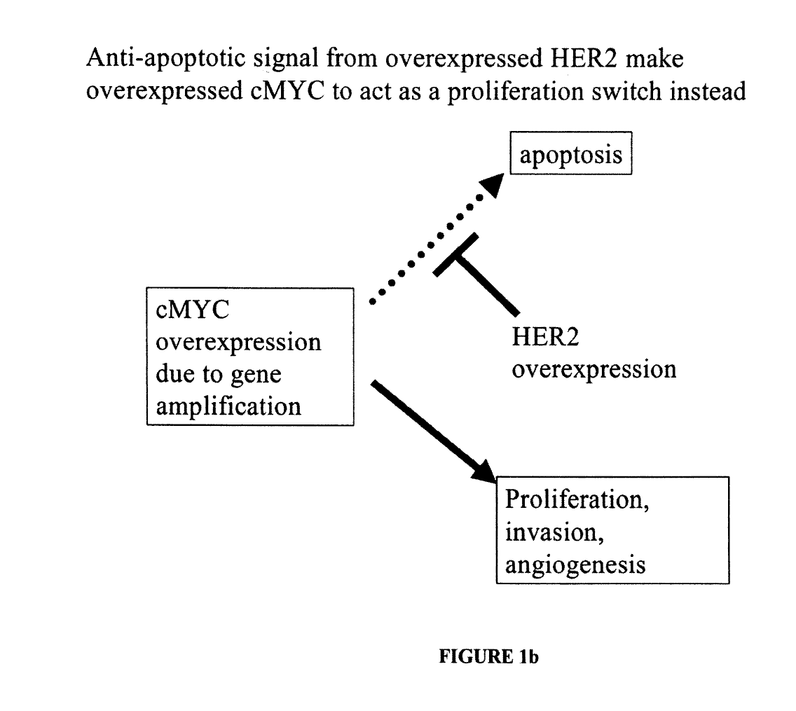

### DESCRIPTION OF THE INVENTION

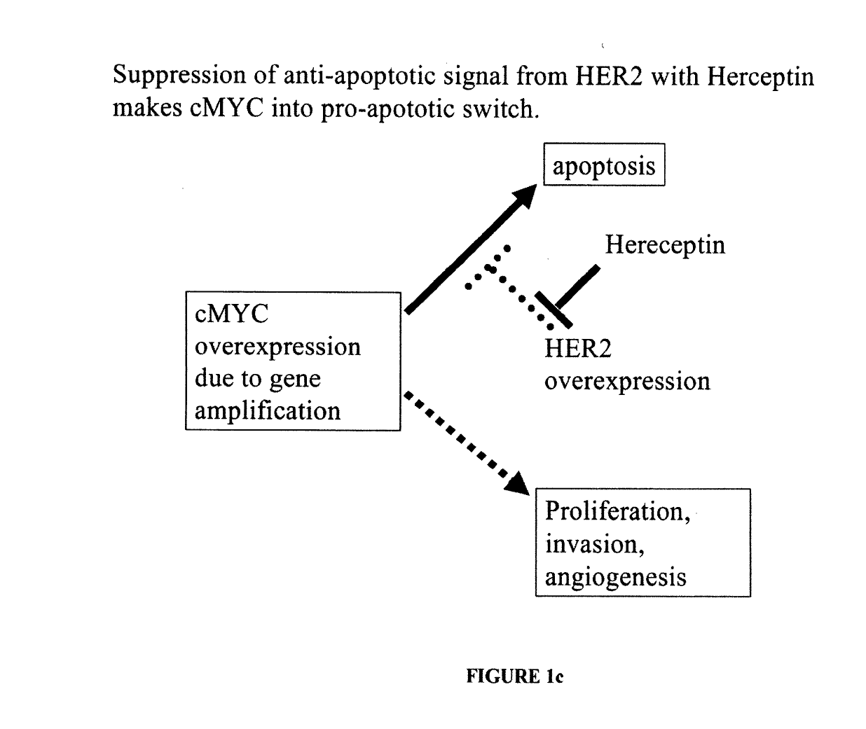

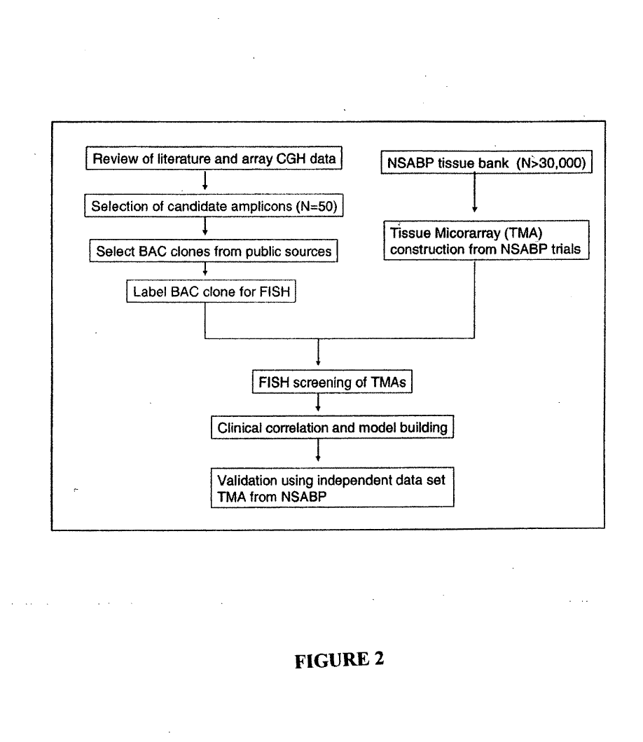

One reason for the high cost of commercially available FISH probes is the cost and difficulty of directly fluorescently labeling bacterial artificial chromosome (“BAC”) clones representing the probes. This disclosure provides a method for fluorescently labeling BAC clones representing known amplicons efficiently by combining a series of whole genome amplification methods and an efficient FISH method for paraffin-embedded tissue that has been archived more than 10 years (see overview in FIG. 2). This labeling and FISH method is a log order less expensive as compared to commercially available probes. Using paraffin block tissue samples for over 30,000 breast and colon cancer cases that are all annotated with clinical follow-up information and treatment received provided a unique source for clinical correlative science studies. Combining the FISH method with tissue microarray (“TMA”) allows screening of more than 100 cases using a single microscopic section, making screening of multiple amplicons in thousands of cases a reality. One of ordinary skill in the art will readily recognize that any number of methods well-known in the art can be used to label probes for FISH applications. Furthermore, because FISH is used to determine amplification, numerous other quantitative or semi-quantitative methods may be used, including, but not limited to, antibody based assays (such as enzyme-linked immunosorbent assay (“ELISA”)) and quantitative PCR (“qPCR”).

### Pilot Project:

In a pilot demonstration project, more than 987 cases from National Surgical Adjuvant Breast and Bowel Project (“NSABP”) trial B-28 were screened comparing 4 cycles of ariamycin (doxorubicin) plus cyclophosphamide (“AC”), versus the same drugs followed by four cycles of paclitaxel (TAXOL®; “ACT”). In this study, tissue microarrays were constructed and FISH assays performed for 10 different in-house developed probes based on array CGH data (two sets are very close to each other, i.e., HER2 and MLN64, APPBP2 and PPM1D). The amplicons and their chromosomal locations are shown as follows:

| | |
|---|---|
| SEB4D | 20q13.32 |
| ZNF217 | 20q13.2 |
| APPBP2 | 17q23.2 |
| TPD52 | 8q21 |
| MLN64 | 17q11-q12 |
| PPM1D | 17q23.2 |
| HER2 | 17q21.1 |
| CYCLIND1 | 11q13 |
| MAL2 | 8q23 |
| C-MYC | 8q24.12-q24.13 |

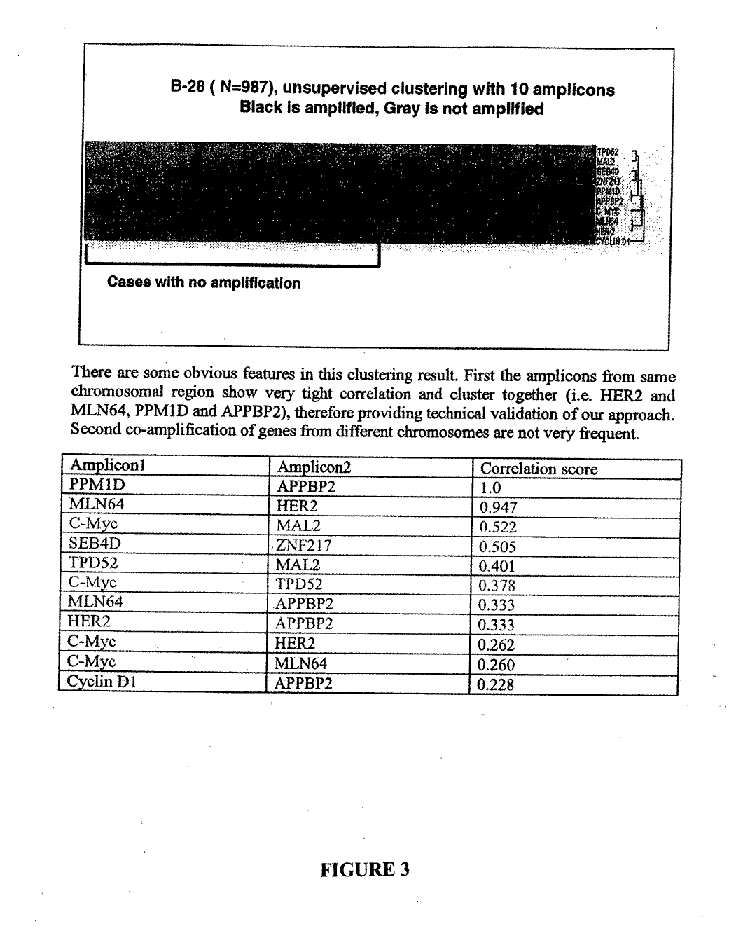

After hybridization of individual probes, cases were scored as either amplified (if signal more than 3 copies per nuclei) or not-amplified (2 copies or less). In order to find the natural class of amplification patterns of these 10 amplicons, non-supervised hierarchical clustering was performed. The results of the pilot study are shown in FIG. 3. What is notable in the result is the close correlation of amplification status of PPMID and APPBP2, and HER2 and MLN6, as expected based on their very close proximity in their chromosomal location. This data proves that the method for BAC labeling as claimed results in highly reproducible results.

In addition, there are cases with no amplification of any of the 10 amplicons. While the proportion of such cases will decrease as more amplicons are screened, it is likely that such subgroups do exist that are relatively resistant to amplification.

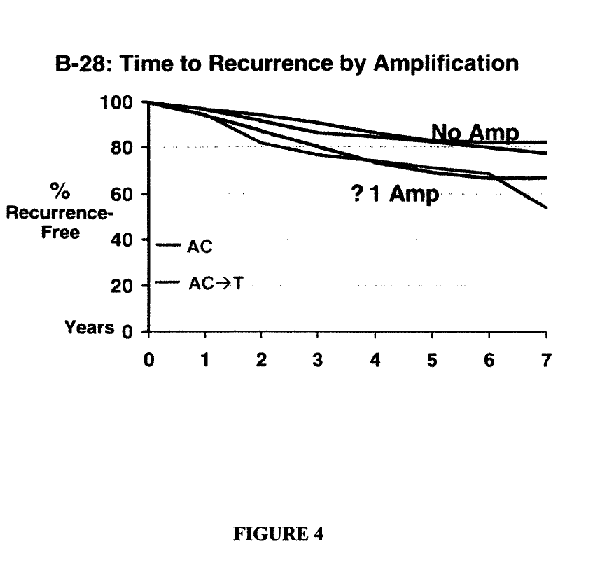

The prognostic value of non-amplification versus any amplification in B-28 according to treatment was examined. Recurrence-free survival of those patients with no amplification of any of the 10 amplicons were significantly better than those with amplification of any of the genes (FIG. 4), while as expected from the nature of the genes in the 10 selected amplicons in this pilot, there was no interaction with the benefit from adding TAXOL® to AC based on amplification phenotype in general in this protocol.

As a result of systematic screening of 27 candidate amplicons that are associated with overexpression (as shown in Table 1), three amplicons (HER2, cMYC, and HTPAP, which is also knows as PPAPDC1B) were identified that are independently prognostic in node-positive breast cancer treated with standard chemotherapy when they are tested in multivariate analysis including other prognostic variables. These three amplicons were identified using the following BACs: PATHVYSION®-HER2 Assay from Vysis, Incorporated; LSI®-C-MYC from Vysis, Incorporated; and HTPAP-RP 11-513D5. Nevertheless, one of ordinary skill in the art would readily recognize multiple other probe sources for the same genes can be utilized with this invention. One of ordinary skill in the art would readily recognize multiple other methods of labeling any probe sources for the same genes can be utilized with this invention. These could include both fluorogenic and chromogenic probe labeling methods.

These 27 amplicons were screened by FISH on TMA constructed from NSABP trial B-28, in which axillary node-positive breast cancer patients were randomly assigned to receive 4 cycles of AC or the same regimen followed by TAXOL® (“ACT”; N=1901). This means that approximately 51,327 FISH assays were performed (27×1901). Selection of the 27 amplicons was based on the following criteria: 1) selected amplicons had been all shown to be associated with moderate to high level of gene expression of the coded genes when amplified in breast cancer tumors or cell lines in the studies conducted by Pollack et al., 2002 (supra) and Hyman et al., 2002 (supra); 2) the public genome sequence map was examined and FISH validated BAC clones were selected that corresponded best with the selected amplicons; and 3) some amplicons, such as MLN64, which were located very close to HER2, were included as an internal control for reproducibility and validity of the assay (that is HER2 and MLN64 amplification were expected to correlate extremely tightly due to their close proximity in chromosome location).

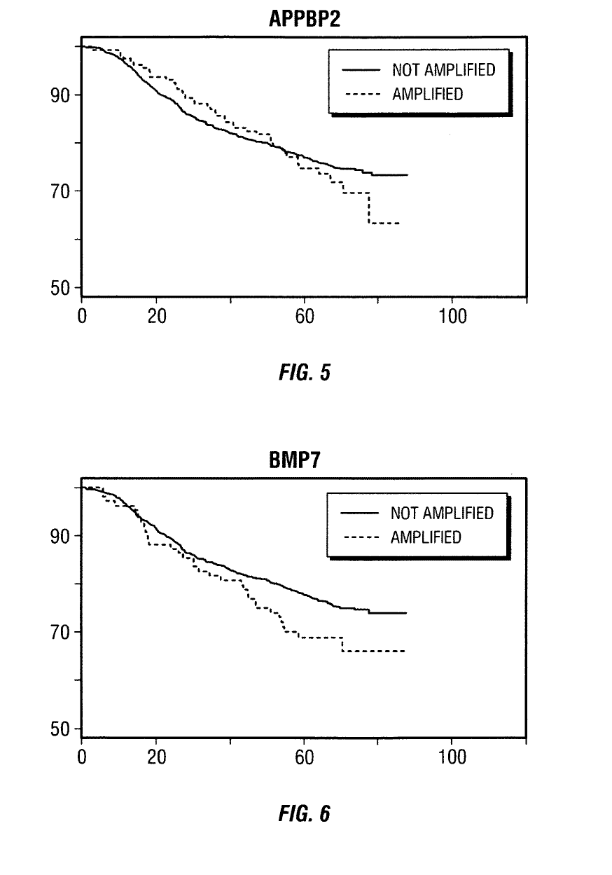

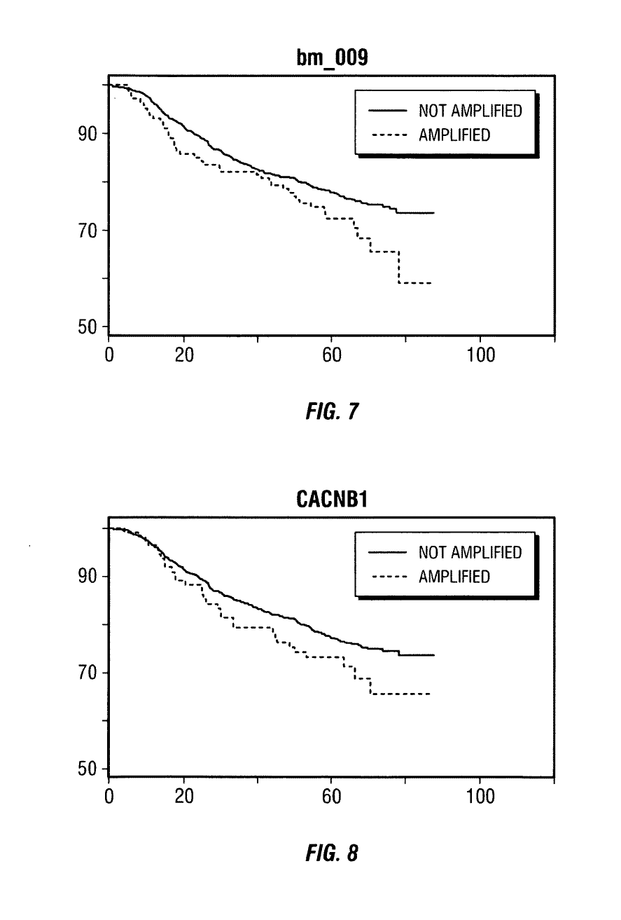

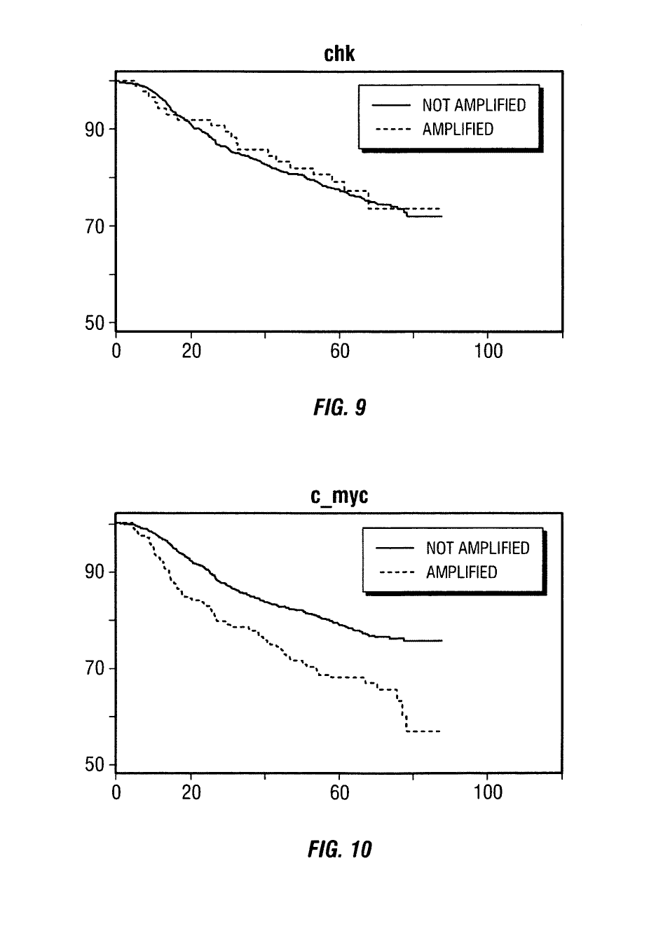

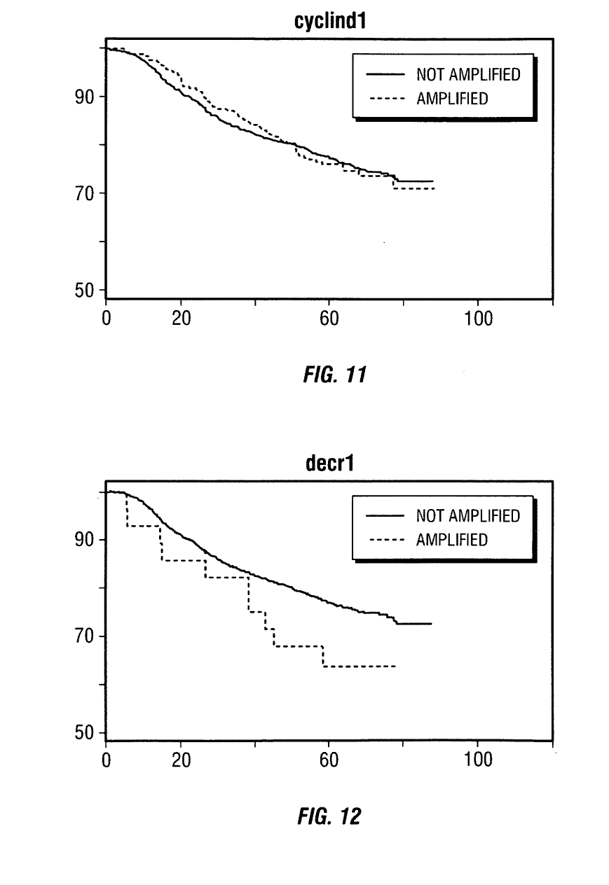

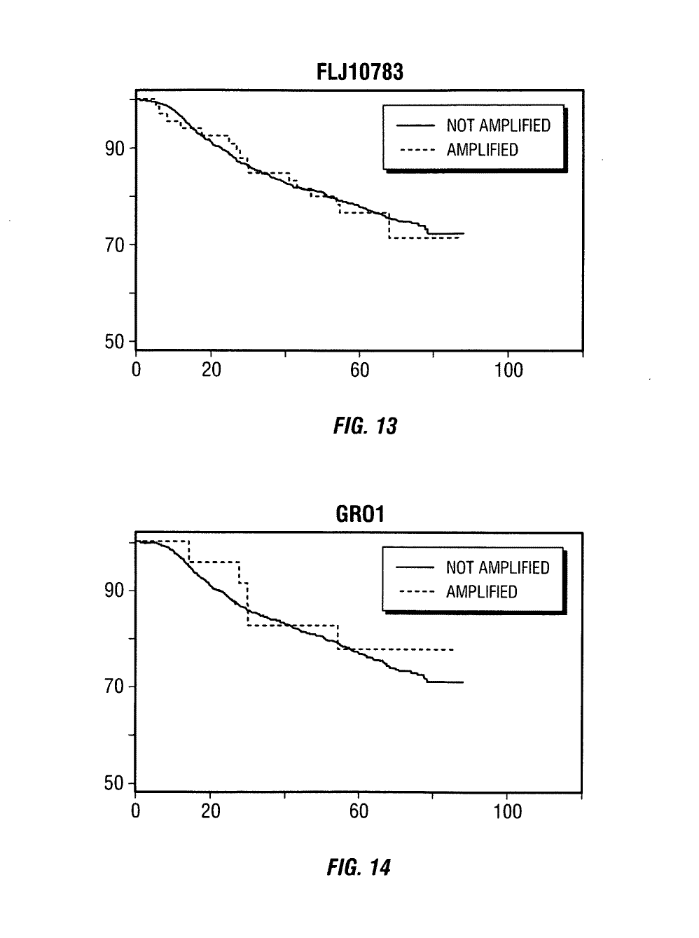

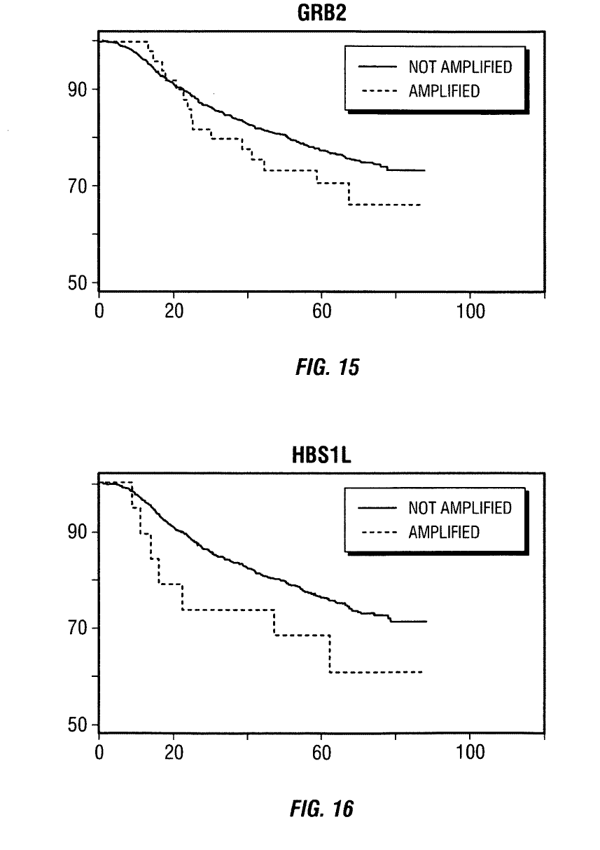

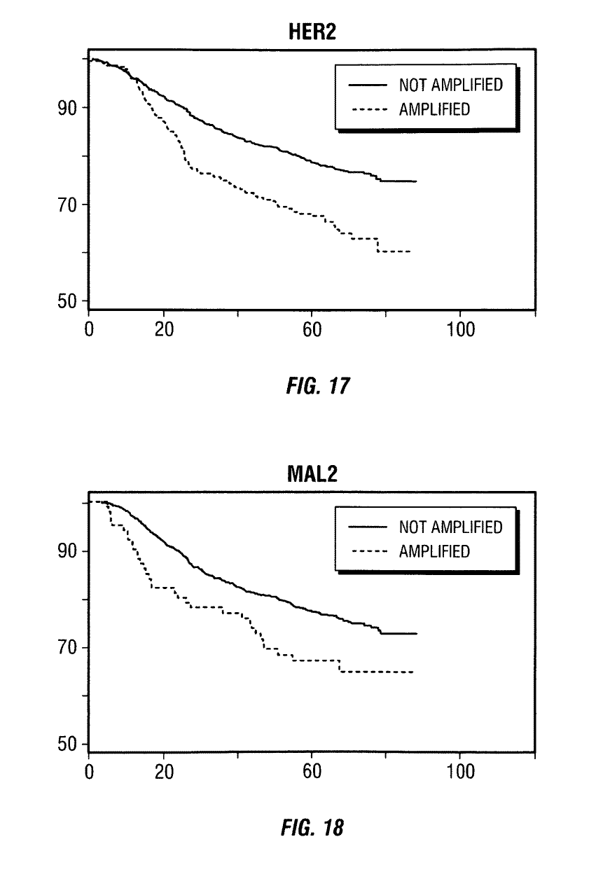

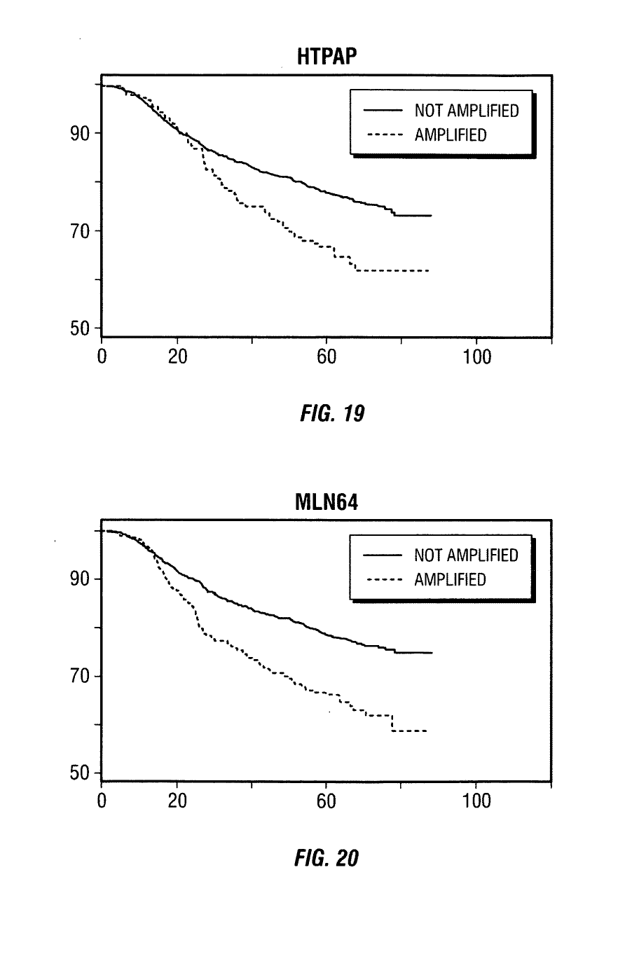

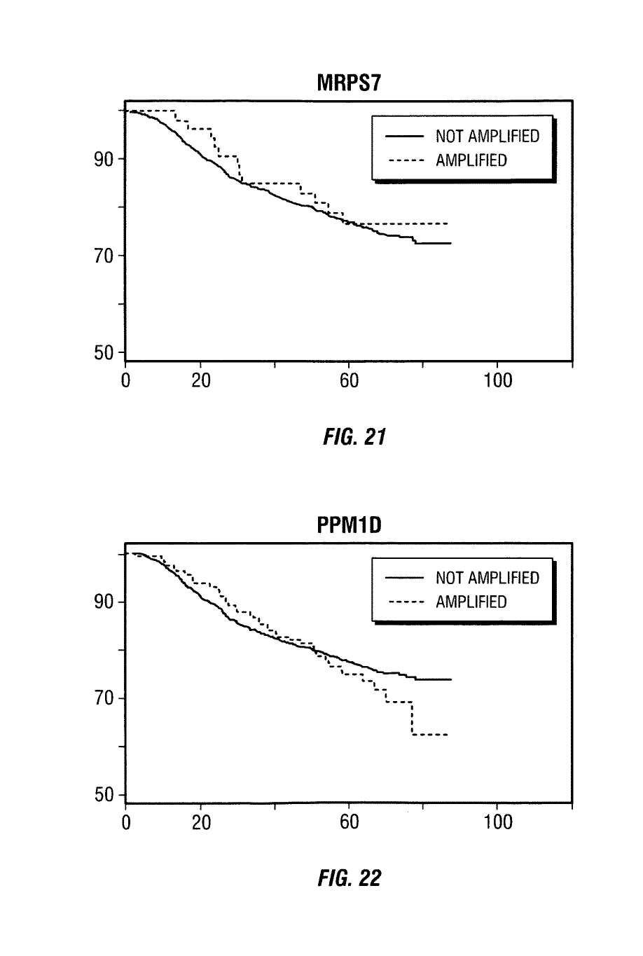

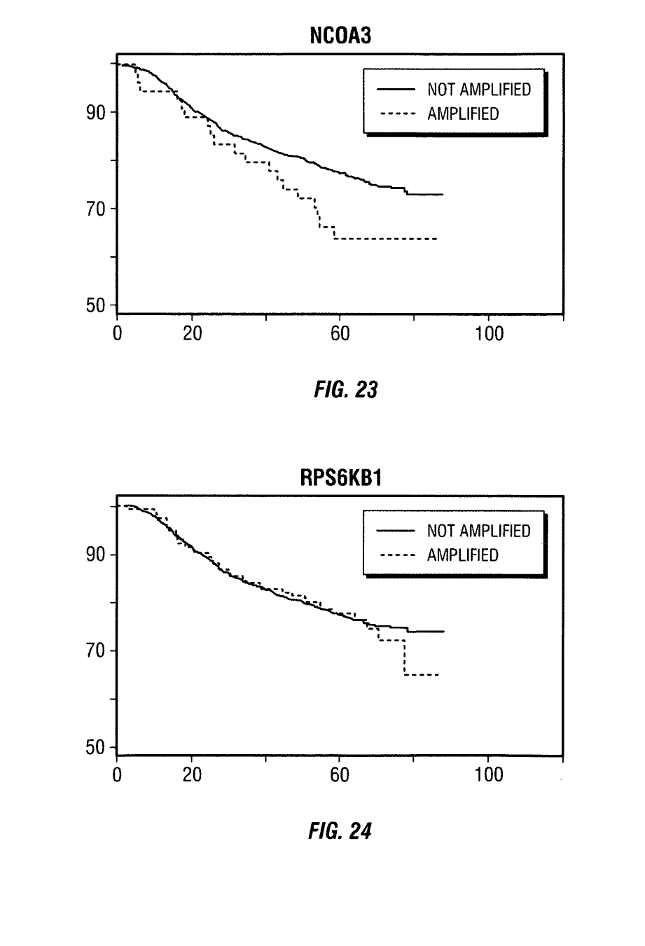

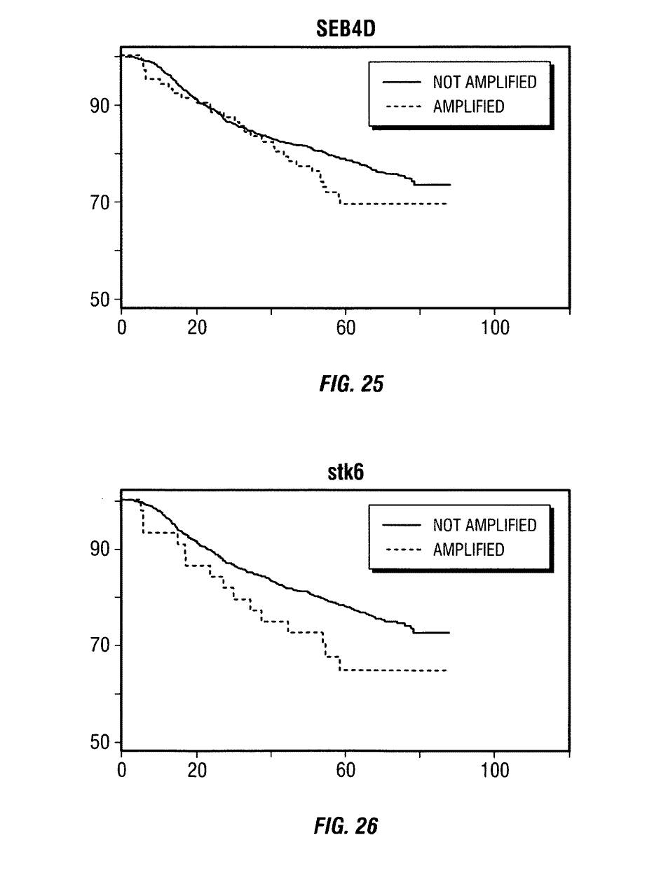

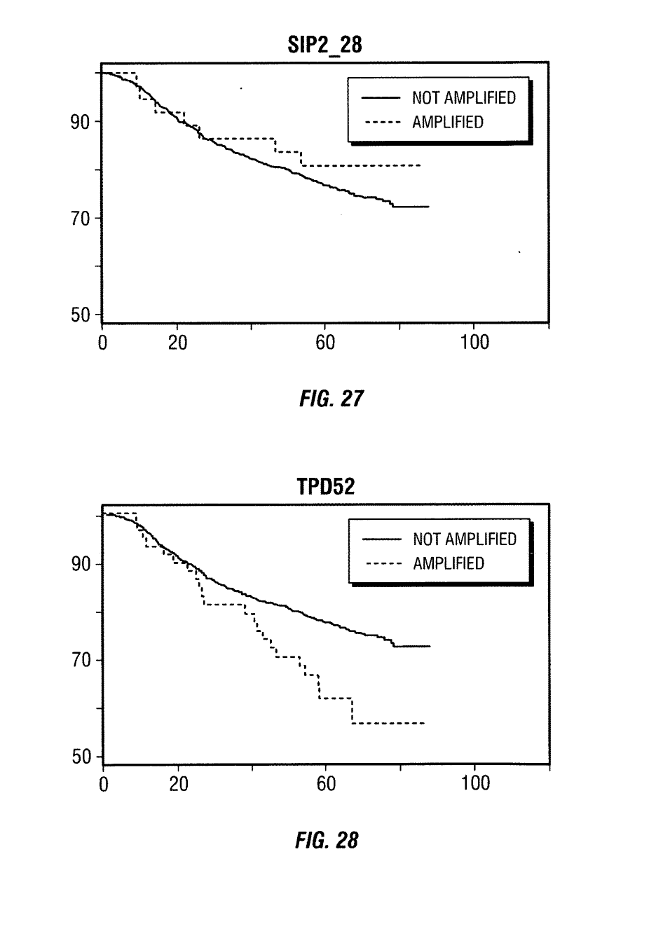

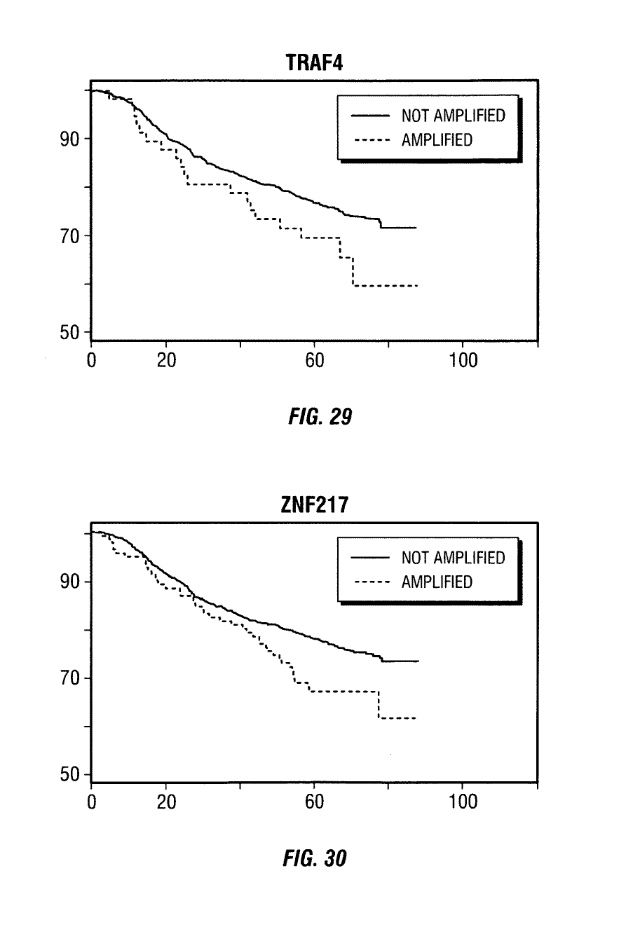

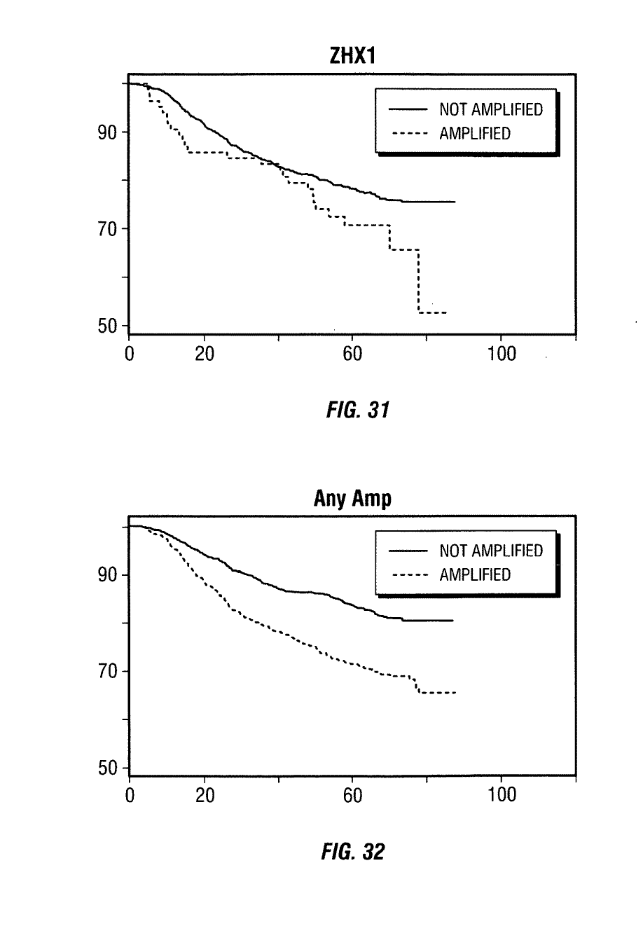

Amplification status was categorized as either amplified or non-amplified, with gene amplification defined as having more than 4 signals (4 dots per single tumor cell nucleus) from in situ hybridization. Correlation with clinical outcome using a univariate Cox proportional hazard model showed that HER2, MLN64 (which is very close to HER2 and highly correlated), cMYC, HTPAP, TPD52, MAL2, and ZNF217 are significantly correlated with clinical outcome of patients entered into the B-28 trial (Table 1). In addition, the presence of any amplification and number of amplifications also showed significant correlation with outcome. Kaplan-Meier plots for each of the 27 amplicons screened are shown in FIGS. 5 to 31. A Kaplan-Meier plot comparing cases with no amplification versus any amplification is shown in FIG. 32.

Multivariate analysis including conventional prognostic markers (tumor size, number of positive nodes, hormone receptor status, and age) was performed. Three amplicons remained significant: HER2; cMYC; and HTPAP (as shown in Table 2).

### HTPAP:

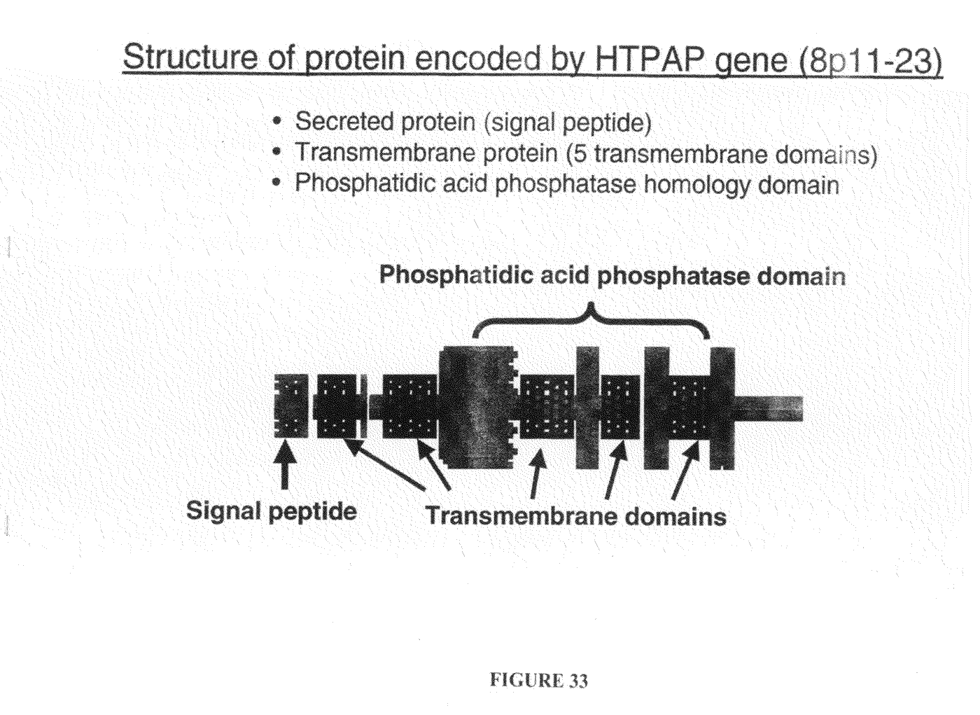

Both HER2 and cMYC have previously been shown to be prognostic in breast cancer. HER2 is the therapeutic target for HERCEPTIN®. However their prognostic role in chemotherapy-treated patients has not been clearly demonstrated. On the other hand, HTPAP is a novel gene that translates into a protein with a phosphatidic acid phosphatase homology domain, a 5′ transmembrane domain, as well as signal peptide that indicates that the protein product is secreted (FIG. 33). The BAC clone used for generation of the FISH probe for HTPAP (clone RP11-513D5) has only three genes in it: HTPAP; WHSC1L1; and DDHD2. Of these, other studies correlating gene amplification with expression in breast cancer cell lines have shown that HTPAP is the one that is overexpressed when this region is amplified (Pollack et al., 2002, supra; Hyman et al., 2002, supra; Ray et al., Cancer Res. 64(1):40-47, 2004). In a review of data from microarray analysis of gene expression in breast cancer, it was reported that HTPAP overexpression is associated with poor prognosis of patients with breast cancer, together with 94 other genes (Jenssen et al., Hum. Genet. 111(4-5):411-420, 2002). These results demonstrate that amplification of the HTPAP gene is an independent prognosticator for breast cancer even after treatment with standard chemotherapy.

While amplification and overexpression of HTPAP in a limited number of breast cancers with 8p11-12 amplification has been described before by other investigators, these studies have not pinpointed HTPAP as the main driver gene in those amplifications since there are other genes that are overexpressed from the region of amplification. By taking advantage of the use of relatively small FISH probes containing only three genes in which HTPAP is the only overexpressed gene, and screening of a large number of cases with defined treatment from a single prospective clinical trial, this disclosure is the first to demonstrate the role of HTPAP as a prognostic factor independent of other prognosticators in breast cancer. Since it is amplified and correlated with poor prognosis even after standard chemotherapy, HTPAP is also an important therapeutic target for breast cancer.

The following characteristics of HTPAP make it an ideal therapeutic and diagnostic target in breast cancer: 1) HTPAP is amplified and a stable clinical diagnostic assay using FISH or PCR can be used to detect the amplification status; 2) it is an independent prognostic factor in heavily treated patients; 3) it is a transmembrane protein with enzyme activity; and 4) it is also secreted. The amplification of this gene being highly correlated with poor prognosis indicates that the blocking of these activities will have beneficial therapeutic effects (as exemplified by the HER2 gene, which has a similar characteristic of being amplified, a prognostic factor, and a cell surface receptor).

Certain embodiments of the present invention include monoclonal antibodies, or a series of monoclonal antibodies, with specificity for the extracellular domain of the HTPAP protein. These antibodies can be used either alone or in combination with chemotherapeutic drugs or antibodies to other targets. The generation of such antibodies can be performed via any number of methods for monoclonal antibody production which are well-known in the art.

In certain embodiments of the present invention, these anti-HTPAP antibodies are used to detect HTPAP protein secreted in the serum, plasma, or a body fluid (such as nipple aspirate from the patients), and compared to normal levels in the diagnosis or monitoring of disease during therapy. Detection may be accomplished by any number of methods well-known in the art, including, but not limited to, radioimmunoassay, flow cytometry, ELISA, or other colormetric assays.

Phosphatidic acid phosphatase domains typically act as an important signaling molecule in cancer cells. Certain embodiments of the present invention include the use of these domains of the HTPAP gene in targeting the development of small molecules that interfere or modulate such activity. Furthermore, the use of antibodies to HTPAP can be used to identify downstream signaling molecules to HTPAP and subsequently targeted by small molecule therapeutics. Certain other embodiments include the blocking of HTPAP gene activity using siRNA, antisense oligonucleotides, or ribozyme approaches that are well-known in the art.

Other genes found to be of marginal prognostic power in this study cohort of AC- or ACT-treated node-positive breast cancer may have significant prognostic power in untreated or node-negative patients—these include TPD52, MAL2, ZNF217, NCOA3, ZHX1, BM_{—}009, BMP7, and STK6, and they also may provide attractive target for therapeutic development. In certain embodiments of the present invention, three prognostic amplified genes, HER2, cMYC, and HTPAP, can be utilized to create a prognostic index to guide treatment decision-making for breast cancer patients. Certain other embodiments include the same three genes together with clinical variables to generate a prognostic index to guide treatment decision-making.

cMYC Predictor:

Cells primed for malignant transformation by cMYC amplification seem to be able to escape the fate of apoptosis with the help of HER2 amplification, however, it is believed that this also makes them dependent on HER2 signaling to survive (FIG. 2b). Therefore inhibition of the HER2 signal by trastuzumab could trigger the pro-apoptotic function of cMYC in such cancer cells (FIG. 2c). This was verified in retrospective analysis of tumor specimens collected as part of NSABP trial B-31, in which patients diagnosed with HER2-overexpressing tumors were randomized to receive chemotherapy or chemotherapy plus HERCEPTIN®. The results of this analysis clearly demonstrated that tumors with co-amplification of both HER2 and cMYC gene are sensitive to trastuzumab.

In an effort to identify clinically important gene amplifications in breast cancer, 27 different commonly amplified genes in breast cancer were screened using FISH. As previously stated, in a unpublished study correlating clinical outcomes of 1900 patients with the status of gene amplification of 27 different genes/loci, HER2, cMYC, and HTPAP were identified as three independent amplified genes that confer a worse prognosis even after standard combination taxane-containing adjuvant chemotherapy. Furthermore, cases that had co-amplification of HER2 and cMYC had much worse prognosis than cases with amplification of either one of the genes.

The status of cMYC in 1344 patients enrolled in the NSABP B-31 trial were examined to test the potential benefits of addition of trastuzumab to chemotherapy in the treatment of patients diagnosed with early stage breast cancer with HER2 gene amplification/overexpression. FISH was used to enumerate the cMYC gene copy number using a commercially available DNA probe (Vysis, Incorporated). Any tumor with more than 10% of cells showing more than 4 copies of cMYC gene was classified as cMYC gene amplified in this analysis. 399 cases out of 1344 total cases studied were classified as cMYC amplified. Tumors with cMYC amplification were believed to be sensitive to inhibition of HER2 signaling due to its activation of a pro-apoptotic signal when the HER2 signal is inhibited by trastuzumab, and that this would translate into a much more significant reduction in the recurrence rate in cMYC amplified cohort in comparison to patients with no amplification of cMYC.

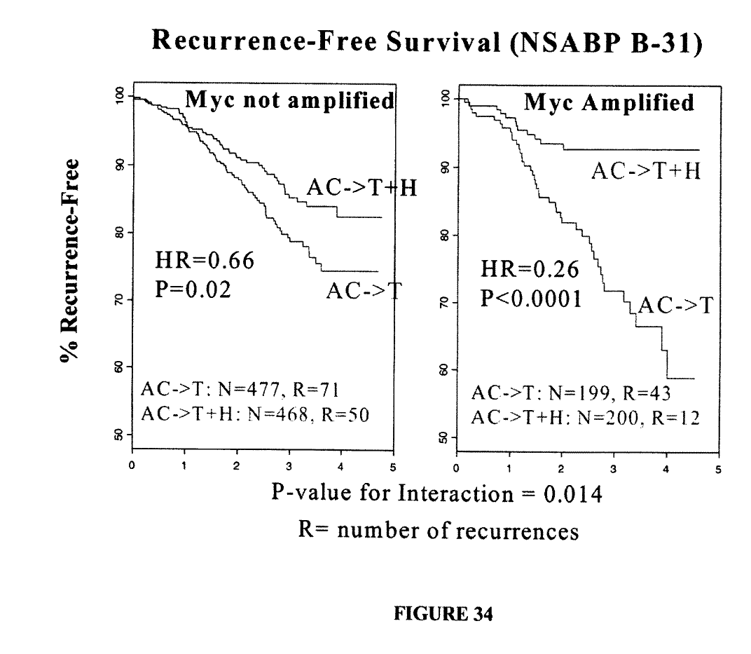

Recurrence-free survival of B-31 patients according to cMYC amplification status is shown in FIG. 34. In patients with no amplification of the cMYC gene (N=945), there was a 34% reduction in recurrence rate when trastuzumab was added to chemotherapy (p=0.02). On the other hand, in patients with cMYC amplification (N=399), there was a 74% reduction in recurrence rate when trastuzumab was added to chemotherapy (p<0.0001). The p-value for the interaction test to determine if this difference between the two cohorts is statistically meaningful was 0.014, thus verifying the cMYC by trastuzumab interaction. In spite of starting with a very poor prognosis, patients with tumors that have co-amplification of HER2 and cMYC end up enjoying near cure of their disease with trastuzumab plus chemotherapy.

Although trastuzumab does not cure all HER2-overexpressing tumors, strategies to add other targeted therapies, such as an inhibitor of angiogenesis, may be useful. However, such an approach is highly toxic and very expensive. cMYC amplified cases should not need additional therapy (other than trastuzumab) due to their sensitivity to trastuzumab. Therefore, one invention of the present disclosure is the screening of patients for approaches that add other targeted therapies to trastuzumab. Furthermore, the present disclosure includes a method of determining a cancer patient's amplification of cMYC and HER2 status. The present disclosure is also applicable to other HER2-targeted therapies since the effect is an indirect one through activation of the pro-apoptotic role of cMYC. In other words, the invention disclosed herein includes methods of determining treatments and treating patients with trastuzumab and other materials based on a patient's cMYC and HER2 status.

In other embodiments, the present invention can be applied in exploiting the pro-apoptotic function of cMYC in cMYC-amplified tumors without HER2 amplification. Instead of directly inhibiting cMYC activity, indirect approaches inhibiting survival signals will likely make such tumors go through programmed cell death by activation of cMYC's pro-apoptotic function.

The test for cMYC in the present disclosure can be either in the format of FISH, quantitative polymerase chain reaction, immunohistochemistry, or other immunological detection method in homogenized tumor tissue, including a single tube, “real-time” quantitative polymerase chain reaction assay that includes HER2, cMYC, HTPAP, and a reference gene to simultaneously detect the presence of amplification of these three genes and provide both prognostic information as well as prediction of response to trastuzumab or other HER2-targeted therapies, as well as the assay and methods of treating a patient based on the results of such an assay.

TABLE 1

| | | | | | Hazard | | Upper |
|---|---|---|---|---|---|---|---|
| Amplicons | Estimate | StdErr | ScoreChiSq | PValue | Ratio | Lower CL | CL |
| any_amplicons | 0.62392 | 0.09873 | 41.234 | <.0001 | 1.866 | 1.538 | 2.265 |
| HER2 | 0.54108 | 0.11464 | 22.8233 | <.0001 | 1.718 | 1.372 | 2.151 |
| MLN64 | 0.55419 | 0.11773 | 22.7323 | <.0001 | 1.741 | 1.382 | 2.192 |
| number_amplicons | 0.07627 | 0.01613 | 22.4652 | <.0001 | 1.079 | 1.046 | 1.114 |
| C_MYC | 0.5156 | 0.12165 | 18.3631 | <.0001 | 1.675 | 1.319 | 2.126 |
| HTPAP | 0.48668 | 0.14443 | 11.5791 | 0.0007 | 1.627 | 1.226 | 2.159 |
| TPD52 | 0.55161 | 0.22118 | 6.3773 | 0.0116 | 1.736 | 1.125 | 2.678 |
| MAL2 | 0.44767 | 0.1837 | 6.0382 | 0.014 | 1.565 | 1.092 | 2.243 |
| ZNF217 | 0.37832 | 0.15995 | 5.6609 | 0.0173 | 1.46 | 1.067 | 1.997 |
| NCOA3 | 0.45526 | 0.23643 | 3.7721 | 0.0521 | 1.577 | 0.992 | 2.506 |
| ZHX1 | 0.39753 | 0.21037 | 3.6178 | 0.0572 | 1.488 | 0.985 | 2.248 |
| BM_009 | 0.30628 | 0.16418 | 3.4934 | 0.0616 | 1.358 | 0.985 | 1.874 |
| BMP7 | 0.32718 | 0.18117 | 3.2904 | 0.0697 | 1.387 | 0.972 | 1.978 |
| STK6 | 0.4405 | 0.26498 | 2.8085 | 0.0938 | 1.553 | 0.924 | 2.611 |
| SEB4D | 0.29955 | 0.19145 | 2.4536 | 0.1173 | 1.349 | 0.927 | 1.964 |
| DECR1 | 0.49841 | 0.32199 | 2.4457 | 0.1179 | 1.646 | 0.876 | 3.094 |
| CACNB1 | 0.27966 | 0.18937 | 2.1866 | 0.1392 | 1.323 | 0.913 | 1.917 |
| TRAF4 | 0.34353 | 0.23789 | 2.1059 | 0.1467 | 1.41 | 0.885 | 2.247 |
| HBS1L | 0.47545 | 0.38277 | 1.572 | 0.2099 | 1.609 | 0.76 | 3.406 |
| GRB2 | 0.2947 | 0.26481 | 1.2395 | 0.2656 | 1.343 | 0.799 | 2.256 |
| PPM1D | 0.13435 | 0.16327 | 0.6779 | 0.4103 | 1.144 | 0.831 | 1.575 |
| SIP2_28 | −0.25338 | 0.38169 | 0.4442 | 0.5051 | 0.776 | 0.367 | 1.64 |
| APPBP2 | 0.10275 | 0.16151 | 0.405 | 0.5245 | 1.108 | 0.808 | 1.521 |
| MRPS7 | −0.10766 | 0.29402 | 0.1342 | 0.7141 | 0.898 | 0.505 | 1.598 |
| GRO1 | −0.14334 | 0.451 | 0.1012 | 0.7503 | 0.866 | 0.358 | 2.097 |
| RPS6KB1 | 0.04841 | 0.17472 | 0.0768 | 0.7817 | 1.05 | 0.745 | 1.478 |
| FUJ10783 | 0.05525 | 0.2567 | 0.0463 | 0.8296 | 1.057 | 0.639 | 1.748 |
| CHK | −0.05045 | 0.23673 | 0.0454 | 0.8312 | 0.951 | 0.598 | 1.512 |
| CYCLIND1 | 0.0266 | 0.13602 | 0.0376 | 0.8463 | 1.027 | 0.787 | 1.341 |

Result of univariate Cox proportional hazard model for each amplicons. Also included is presence of any amplification and number of amplification.

TABLE 2
Multivariate Cox model including clinical
variables and HER2, cMYC, and HTPAP.

| | | | Hazard ratio |
|---|---|---|---|
| Variable | Chi-Square | P value | (95% CI) |
| Treatment (AC vs ACT) | 2.7084 | 0.0998 | 0.854 (0.708-1.031) |
| Positive node (4-9) | 33.6884 | <.0001 | 1.844 (1.500-2.268) |
| Positive node (=>10) | 34.2087 | <.0001 | 2.852 (2.008-4.053) |
| medium tumor size | 2.2142 | 0.1367 | 1.169 (0.952-1.436) |
| large tumor size | 14.8289 | 0.0001 | 1.761 (1.320-2.348) |
| young_ER_negative | 30.8256 | <.0001 | 2.131 (1.631-2.783) |
| old_ER_positive | 3.8946 | 0.0484 | 0.775 (0.602-0.998) |
| old_ER_negative | 7.5734 | 0.0059 | 1.499 (1.124-2.000) |
| intermediate_grade | 5.8421 | 0.0156 | 1.898 (1.129-3.192) |
| poor_grade | 8.532 | 0.0035 | 2.161 (1.289-3.624) |
| missing_grade | 2.0411 | 0.1531 | 1.653 (0.830-3.293) |
| HER2 | 3.4508 | 0.0632 | 1.245 (0.988-1.569) |
| cMYC | 4.7202 | 0.0298 | 1.307 (1.027-1.665) |
| HTPAP | 14.2542 | 0.0002 | 1.726 (1.300-2.292) |

What is claimed:

1. A method of treating breast cancer comprising:
measuring the expression levels or amplification of HTPAP in a patient having breast cancer;
providing a patient having increased levels of HTPAP expression or HTPAP amplification with therapeutic quantities of at least one compound that interferes with the phosphatidic acid phosphatase activity of HTPAP.

2. The method of claim 1 wherein the expression levels of HTPAP is measured via a technique selected from the group consisting of: an enzyme-linked immunosorbent assay; radioimmunoassay; and flow cytometery.

3. The method of claim 2 wherein the enzyme-linked immunosorbent assay is performed on supemant from the patient to measure soluble HTPAP protein concentrations.

4. The method of claim 1 wherein the expression levels of HTPAP is measured via a real time quantitative polymerase chain reaction assay.

5. The method of claim 1 wherein the at least one compound is an anti-HTPAP specific antibody.

6. The method of claim 5 wherein the anti-HTPAP specific antibody is a humanized monoclonal antibody.

7. The method of claim 1 wherein the HTPAP amplification is measured via fluorescent in situ hybridization.

8. A method of monitoring the breast cancer treatment comprising measuring the expression levels or amplification of HTPAP in a patient having breast cancer wherein decreasing quantities of HTPAP is indicative of beneficial treatment.

9. The method of claim 8 wherein the expression levels of HTPAP is measured via a technique selected from the group consisting of: an enzyme-linked immunosorbent assay; radioimmunoassay; and flow cytometery.

10. The method of claim 9 wherein the enzyrne-linked immunosorbent assay is performed on supemant from the patient to measure soluble HTPAP protein concentrations.

11. The method of claim 8 wherein the expression level of HTPAP is measured via a real time quantitative polymerase chain reaction assay.

12. The method of claim 8 wherein the HTPAP amplification is measured via fluorescent in situ hybridization.

13. A method of treating breast cancer comprising:
screening a breast cancer patient for amplification of the cMYC gene; and
treating a patient having amplification of the cMYC gene with therapeutic quantities of a compound that interferes with HER2 signaling.

14. The method of claim 13 wherein the compound that interferes with HER2 signaling is Trastuzumab.

15. The method of claim 13 wherein screening for amplification of the cMYC gene is done via fluorescent in situ hybridization with a sample of the cancer tissue.

16. The method of claim 13 further comprising screening the breast cancer patient for amplification of the HER2 gene.

17. The method of claim 16 wherein screening for amplification of the HER2 gene is done via fluorescent in situ hybridization with a sample of the cancer tissue.

18. The method of claim 16 wherein the therapeutic quantities of a compound that interferes with HER2 signaling are used to treat a patient having amplification of both the cMYC and HER2 genes.

19. The method of claim 13 further comprising treating the patient with chemotherapy in conjunction with the compound that interferes with HER2 signaling.

20. The method of claim 13 wherein screening for amplification of the cMYC gene is done via fluorescent in situ hybridization with a sample of the cancer tissue.

21. A method of screening for markers useful in predicting the efficacy of a specified cancer comprising:
constructing a tissue microarray from a tissue bank comprising multiple tissue samples that are annotated with clinical follow-up data;
labeling polynucleic acid probes specific for oncogenes or cancer associated genes known to be potential amplicons;
performing fluorescent in situ hybridization analysis on the tissue microarray; and
correlating the result of the fluorescent in situ hybridization with the clinical follow-up data.
